(19) United States
(12) Patent Application Publication
(10) Pub. No.: US 2020/0147172 A1
(43) Pub. Date: May 14, 2020

(54) USE OF CREG IN TREATMENT OF NONALCOHOLIC FATTY LIVER DISEASE AND TYPE 2 DIABETES

(71) Applicant: GENERAL HOSPITAL OF CHINESE PLA NORTHERN THEATER COMMAND, Shenyang, Liaoning (CN)

(72) Inventors: Yaling HAN, Shenyang, Liaoning (CN); Quanyu ZHANG, Shenyang, Liaoning (CN); Chenghui YAN, Shenyang, Liaoning (CN); Xiaoxiang TIAN, Shenyang, Liaoning (CN)

(21) Appl. No.: 16/474,903

(22) PCT Filed: Dec. 21, 2017
(86) PCT No.: PCT/CN2017/117790

§ 371 Date: Jun. 28, 2019

(30) Foreign Application Priority Data
Dec. 30, 2016 (CN) 201611254504.1

(51) Int. Cl. A61K 38/17; C12N 15/85; A61P 3/10; A01K 67/027

(52) U.S. Cl. CPC A61K 38/1709; C12N 15/85; A61P 3/10; A01K 67/0276; A01K 2217/07; A01K 2227/105; A01K 2267/0362; A01K 2217/05; A01K 2207/15

(57) ABSTRACT

The present invention relates to a use of cellular repressor of E1A-stimulated genes (CREG) protein, and in particular to a use of a CREG protein or an active fragment thereof in manufacture of a medicament for the prevention and/or treatment of a fatty liver disease and type 2 diabetes. The present invention also relates to a use of a recombinant vector or recombinant cell expressing a CREG protein or an active fragment thereof in manufacture of a medicament for the prevention and/or treatment of a fatty liver disease and type 2 diabetes. The invention also relates to a corresponding kit, such as a kit used for the predication and/or evaluation of therapeutic effect and prognosis of a fatty liver disease and type 2 diabetes.

### CROSS-REFERENCE TO RELATED APPLICATIONS

The present invention claims priority to Chinese Patent Application No. 201611254504.1, entitled “Use of CREG in Treatment of Nonalcoholic Fatty Liver and Type 2 Diabetes”, filed on Dec. 30, 2016.

### TECHNICAL FIELD

The invention belongs to a medical use of CREG gene, in particular to a use of cellular repressor of E1A-stimulated genes (CREG) in the treatment of a nonalcoholic fatty liver disease and a type 2 diabetes, specifically to a use of a CREG preparation in manufacture of a medicament for the prevention, alleviation and/or treatment of a fatty liver (especially a nonalcoholic fatty liver disease) and/or a type 2 diabetes.

### BACKGROUND ART

Fatty liver, as a pathological metabolic state of liver, refers to a lesion in which excessive accumulation of fat in liver cells is caused by various reasons. Fatty liver is generally induced by a variety of diseases and is a comprehensive manifestation of adverse effects of many liver diseases on liver cells. Patients with light clinical manifestations may have no symptoms, while their life with moderate or severe manifestations may be threatened. According to whether there exists a history of excessive drinking, the fatty liver is divided into two types: “alcoholic fatty liver disease (ALD)” and “non-alcoholic fatty liver disease (NAFLD)”. NAFLD and ALD are different in aspects of pathogenic factors, natural outcomes, and prognosis, so that their treatment strategies are also different. NAFLD has become the first major chronic liver disease in developed countries in Europe and America and in rich areas of China. In China, the population of patients with NAFLD ranks only second to that with hepatitis B. At present, NAFLD caused by unhealthy lifestyles as well as cirrhosis and liver cancers induced thereby are seriously threatening the health of people of all ages in China, and are also recognized as a common cause of concealed cirrhosis.

NAFLD specifically refers to a clinicopathological syndrome of fat accumulation in liver cells that is not caused by alcohol and other defined liver damages. In addition to directly leading to cirrhosis and hepatocellular carcinoma, NAFLD can also affect the progression of other chronic liver diseases and participate in the pathogenesis of atherosclerosis in type 2 diabetes and cardiovascular and cerebrovascular diseases. NAFLD has become a new challenge to human health and medical research in recent years. The treatment of NAFLD is currently basic support therapy, losing fat and losing weight, insulin sensitizer, reducing blood fat, hepatoprotective drugs, etc., but there is still no effective protective drug.

Type 2 diabetes is non-insulin dependent diabetes. The cause of type 2 diabetes is heredity, obesity, high-calorie diet, insufficient physical activity, and the like. The main manifestation of type 2 diabetes is a decrease in insulin sensitivity. Diabetes as a chronic multiple disease has now become a major public health issue of worldwide concern. The number of people with diabetes in China has already ranked the first in the world. However, in addition to insulin replacement therapy, the treatment of diabetes includes various types of oral drugs such as biguanides and sulfonylureas. There are currently no genetic modulation and therapeutic agents for the treatment of diabetes.

Cellular repressor of E1A-stimulated gene (CREG) is a small-molecular-weight secreted glycoprotein that is widely expressed in mature tissue cells and has important physiological functions for maintaining tissue and cell maturation and differentiation. The CREG protein consists of 220 amino acids and contains 3 mannose-6-phosphate (M6P) glycosylation sites that interact with M6P receptor. CREG is widely expressed in various types of organism cells, can regulate the expression and activation of various cytokines such as interleukins and tumor necrosis factors, and has also been found to be associated with inflammatory activity caused by obesity. It is a key molecule regulating glycoprotein metabolism pathways and abnormal inflammatory responses.

### DESCRIPTION OF THE INVENTION

In one aspect, the invention provides a novel use of a target gene CREG for the treatment of a non-alcoholic fatty liver disease and type 2 diabetes.

In another aspect, the invention provides a use of a CREG gene for the treatment of a non-alcoholic fatty liver disease and type 2 diabetes, wherein the treatment aims at a plurality of downstream targets of the CREG gene.

In one embodiment, the present invention used a wild-type C57 mouse and a CREG liver-specific gene-knockout mouse as experimental subjects, and the function of the CREG gene was studied by a diet induced obesity (DIO) mouse model induced by a high-fat diet. The results showed that compared with the wild-type (WT) mice, the CREG gene-knockout (KO) mice showed obesity, their body weight was significantly higher than that of WT mice fed with the same diet, and the weight and fasting blood glucose level of the CREG gene KO mice were higher than those of the control WT mice, and the liver function of the CREG gene KO mice was significantly worse than that of the WT mice. The inventors further found in an intraperitoneal injection glucose tolerance test that the CREG gene KO mice had a significantly decreased tolerance of glucose. The general appearance of liver, liver weight, liver/body weight ratio in the mice and the results of pathological staining of lipid components all indicated that the fatty liver disease of the CREG-KO mice in the HFD (high fat diet) group was significantly severe, the lipid accumulation significantly increased, and the indicators such as blood glucose levels and glucose tolerance become significantly worse (FIG. 5), while as compared to WT group, the CREG transgenic mice group (CREG-TG group) with high expression of CREG showed a significantly reduced liver steatosis and obviously improved indicators such as blood glucose level and glucose tolerance under HFD conditions. These results indicate that the knockout of CREG gene exacerbates the development of fatty liver disease and type 2 diabetes; while the overexpression of CREG gene may inhibit the development of fatty liver disease and type 2 diabetes (through the JNK1 signaling pathway, FIG. 7).

The inventors' research proves that CREG has the functions of inhibiting obesity, lowering blood glucose level, reducing liver lipid accumulation, protecting liver function, especially improving the control of fatty liver and type 2 diabetes in a model with high fat-induced fatty liver and type 2 diabetes.

In another aspect, the invention relates to a use of a CREG protein or an active fragment thereof in manufacture of a medicament for the prevention and/or treatment of a fatty liver disease and a type 2 diabetes.

In still another aspect, the present invention also relates to a use of a nucleic acid molecule encoding a CREG protein or an active fragment thereof, a downstream regulatory target gene thereof, a recombinant vector or a recombinant cell expressing the CREG protein or an active fragment thereof, in manufacture of a medicament, wherein the medicament is used for the prevention and/or treatment of a fatty liver disease and a type 2 diabetes.

In an embodiment of the invention, the recombinant vector comprises a nucleic acid molecule encoding a CREG protein or an active fragment thereof.

In another aspect, the present invention relates to a use of an agent for detecting an expression level of a CREG protein or an active fragment thereof in manufacture of a kit, wherein the kit is used for the predication and/or evaluation of therapeutic effect and prognosis of a fatty liver disease and type 2 diabetes.

In yet another aspect, the invention also relates to a use of a CREG protein or an active fragment thereof for screening for a medicament for the prevention and/or treatment of a fatty liver disease and type 2 diabetes.

In an embodiment of the present invention, the CREG protein or an active fragment thereof can be used as a target protein for screening for a medicament for the prevention and/or treatment of a fatty liver disease and type 2 diabetes; for example, an agent for promoting upregulation of the CREG protein or an active fragment thereof can be used as a medicament for the prevention and/or treatment of a fatty liver disease and type 2 diabetes.

In another aspect, the invention relates to a composition comprising a CREG protein or an active fragment thereof, a nucleic acid molecule encoding a CREG protein or an active fragment thereof, a recombinant vector or recombinant cell expressing a CREG protein or an active fragment thereof, and optionally a pharmaceutically acceptable carrier or excipient, wherein the composition is used for the prevention and/or treatment of a fatty liver disease and type 2 diabetes.

In yet another aspect, the invention also relates to a kit comprising an agent for detecting an expression level of a CREG protein or an active fragment thereof, wherein the kit is used for the prediction and/or evaluation of therapeutic effect and prognosis of a fatty liver disease and type 2 diabetes.

In another aspect, the invention relates to a use of a CREG protein or an active fragment thereof for the prevention and/or treatment of a fatty liver disease and type 2 diabetes.

In yet another aspect, the invention also relates to a CREG protein or an active fragment thereof, which is used for the prevention and/or treatment of a fatty liver disease and type 2 diabetes.

In another aspect, the present invention relates to a use of a nucleic acid molecule encoding a CREG protein or an active fragment thereof, a downstream regulatory target gene thereof, a recombinant vector or recombinant cell expressing the CREG protein or an active fragment thereof, in the prevention and/or treatment of a fatty liver disease and type 2 diabetes.

In one embodiment, the downstream regulatory target gene of the invention is JNK1.

In still another aspect, the present invention also relates to a nucleic acid molecule encoding a CREG protein or an active fragment thereof, a downstream regulatory target gene thereof, and a recombinant vector or recombinant cell expressing a CREG protein or an active fragment thereof, which is used for the prevention and/or treatment of a fatty liver disease and type 2 diabetes. In an embodiment of the invention, the downstream regulatory target gene of the invention is JNK1.

In another aspect, the invention relates to a method of preventing and/or treating a fatty liver disease and type 2 diabetes, comprising administering to a subject a therapeutically effective amount of a CREG protein or an active fragment thereof.

In a further aspect, the present invention relates to a method of preventing and/or treating a fatty liver disease and type 2 diabetes, comprising administering to a subject a therapeutically effective amount of a nucleic acid molecule encoding a CREG protein or an active fragment thereof, a downstream regulatory target gene thereof, a recombinant vector or recombinant cell expressing a CREG protein or an active fragment thereof. In an embodiment of the invention, the downstream regulatory target gene of the invention is JNK1.

In another aspect, the present invention is also a method of preventing and/or treating a fatty liver disease and type 2 diabetes, comprising administering to a subject a therapeutically effective amount of a substance that modulates an expression level of a CREG gene or a CREG protein.

In an embodiment of the invention, the subject is a mammal, optionally selected from the group consisting of a rat, a mouse, a dog, a pig, a monkey, a human.

In an embodiment of the invention, the CREG protein is a recombinant CREG protein derived from a mammal, in particular from a human. In an embodiment of the invention, the GenBank number of the CREG protein is NP_003842.1. In an embodiment of the invention, the GenBank number of the CREG gene is NM_003851.2.

In an embodiment of the present invention, the active fragment of the CREG protein refers to a fragment having a function of a CREG protein, which may be a portion of the CREG protein, or may be a fragment obtained by deleting, adding or substituting an amino acid sequence of the CREG protein. The methods for preparing or obtaining the active fragment of the CREG protein are well known in the art, for example, the active fragment is a fragment comprising a portion of the CREG protein that binds to a ligand or receptor, or a fragment that remains a function of the CREG protein after deletion, addition or substitution of an amino acid. It is well known to those skilled in the art that some key amino acids on the CREG protein are closely related to its activity, and the mutation may affect the activity of the protein. For example, the activity and function of the CREG protein are influenced when the lysines at positions 136 and 137 of the CREG protein are mutated to alanine, or the amino acids at positions 141-144 of the CREG protein are subjected to deletion mutations (Sacher M, PNAS, 2005; 102 (51): 18326-18331). Those skilled in the art may avoid the above-mentioned sites that may affect the activity as needed, and perform modifications such as deletion, addition or substitution on the other sites, so that the modified CREG protein still has the activity or function of the CREG protein.

In an embodiment of the present invention, the prevention and/or treatment of a fatty liver disease and type 2 diabetes means inhibiting or retarding the occurrence of the fatty liver disease and type 2 diabetes, inhibiting or retarding the occurrence of a disease related to the fatty liver disease and type 2 diabetes.

In an embodiment of the invention, using the detection of an expression level of a CREG protein or an active fragment thereof in prediction and/or evaluation refers to the prediction of occurrence of a fatty liver disease and type 2 diabetes, or the evaluation of its therapeutic effect or prognosis when the expression level of the CREG protein or an active fragment thereof in blood, tissue or cells is lower than a reference value.

In an embodiment of the invention, the mammal may be, for example, a rat, a mouse, a dog, a miniature pig, a monkey, a human, or the like.

In an embodiment of the invention, the expression level of the CREG protein or an active fragment thereof can be detected by a method well known in the art, such as by amplification of CREG mRNA by polymerase chain reaction and quantitative reaction, or by Western Blot to detect the expression level of the CREG protein.

In an embodiment of the invention, the expression level of the protein refers to the mRNA level or the protein level.

In an embodiment of the invention, the up-regulating/down-regulating the expression of a protein in a tissue/cell means increasing or decreasing by at least 20%, 30%, 40%, 50%, 60%, 70%, 80%, 90%, 100%, or increasing by greater than 100%, of the protein level or mRNA level in the tissue/cell. The up-regulating or down-regulating described therein is compared to the uninterrupted tissues/cells (e.g., tissues/cells transfected with control vectors).

The present invention has the following advantages and effects over the prior art:

(1) The present inventors have discovered a novel function of the CREG gene, that is, the CREG gene has an effect of improving the control of fatty liver disease and type 2 diabetes.

(2) Based on the protective effects of CREG in the prevention and treatment of the fatty liver disease and type 2 diabetes, it can be used in manufacture of a medicament for the prevention, alleviation and/or treatment of the fatty liver disease and/or type 2 diabetes.

It is known to those skilled in the art that the technical solution of the present invention does not necessarily achieve the above effects simultaneously, as long as any of the technical effects or any combination thereof can be realized.

### BRIEF DESCRIPTION OF THE DRAWINGS

FIG. 1 shows a schematic diagram of targeting strategy for the construction of CREG conditional knockout mice.

FIG. 2 shows overexpression of CREG transgene.

FIGS. 3A to 3D show the results of body weights and fasting blood glucose levels in WT and CREG-KO mice; FIG. 3A shows a histogram of comparison of mouse body weights, FIG. 3B shows a histogram of comparison of fasting blood glucose levels; FIG. 3C shows a trend chart of body weights, and FIG. 3D shows a trend chart of blood glucose levels (*: p<0.05 vs WT-NC group, #: p<0.01 vs KO-NC group).

FIGS. 3E to 3H show the results of body weights and fasting blood glucose levels in NTG (transfected empty vector group) and CREG-TG mice; FIG. 3E shows a histogram of comparison of mouse body weights, FIG. 3F shows a histogram of comparison of fasting blood glucose levels; FIG. 3G shows a trend graph of body weights, and FIG. 3H shows a trend graph of blood glucose levels (*: p<0.05 vs NTG-NC group, #: p<0.01 vs TG-NC group).

FIGS. 4A to 4B show the results of intraperitoneal injection glucose tolerance in WT and CREG-KO mice; FIG. 4A shows a statistical chart of blood glucose levels at different time points after intraperitoneal injection of glucose in mice, and FIG. 4B shows a comparison chart of the area under curve (AUC) of glucose tolerance of each group of mice (*: p<0.01 vs WT-HFD group).

FIGS. 4C to 4D show the result graphs of intraperitoneal injection glucose tolerance in NTG and CREG-TG mice; FIG. 4C shows a statistical chart of blood glucose levels at different time points after intraperitoneal injection of glucose in mice, and FIG. 4D shows a comparison chart of the area under curve (AUC) of glucose tolerance of each group of mice (*: p<0.01 vs NTG-HFD group).

FIG. 5 shows a result graph of liver ultrasonic test and a histogram of liver/body weight ratio in CREG-KO and WT mice; A shows a result graph of liver ultrasonic test, B shows a histogram of liver/body weight ratio in CREG-KO mice (*: p<0.05 vs WT-NC group, #: p<0.01 vs KO-NC group), and C shows a histogram of liver/body weight ratio in CREG-TG mice (#: p<0.01 vs KO-NC group).

FIG. 6 shows HE and oil red 0 staining results in WT, CREG-KO, NTG and CREG-TG mice.

FIG. 7 shows that the CREG gene plays a role in improving hepatic steatosis and type 2 diabetes by regulating the JNK1 signaling pathway. A shows a chart of changes in body weight, B shows a chart of changes in liver weight/body weight, C shows a chart of changes in blood glucose levels, and D shows an HOMA-IR evaluation chart (*: p<0.05 vs WT-HFD group, #: p<0.01 vs CREG-CKO-HFD group).

### DETAILED EMBODIMENTS OF THE INVENTION

The embodiments of the present invention will be described in detail below with reference to the examples, however, those skilled in the art would understand that the following examples are intended to illustrate the invention and are not intended to limit the scope of the invention. Those without specific conditions in the examples are carried out according to the conventional conditions or the conditions recommended by the manufacturers. The reagents or instruments without giving manufacturers are conventional products that can be obtained commercially.

The experimental data of the present invention are all represented as a percentage. The two-sample rates were compared using the chi-square test, and the statistical analysis was performed using the SPSS 19.0 software package. There exists a statistical difference at p<0.05.

Experimental Animals and Breeding Thereof

Species, gender, age and source of experimental animals: C57BL/6 (WT) mice and CREG-KO mice or CREG-highly expressed transgenic (i.e., CREG-TG) mice, male, 8 weeks old. C57BL/6 mice were purchased from Beijing Huafukang Biotechnology Co., Ltd.

CREG Gene-Knockout (Transgenic) Mice

1. Construction of CREG Conditional Knockout Mice (KO)

1.1 Gene Information and Vector Design

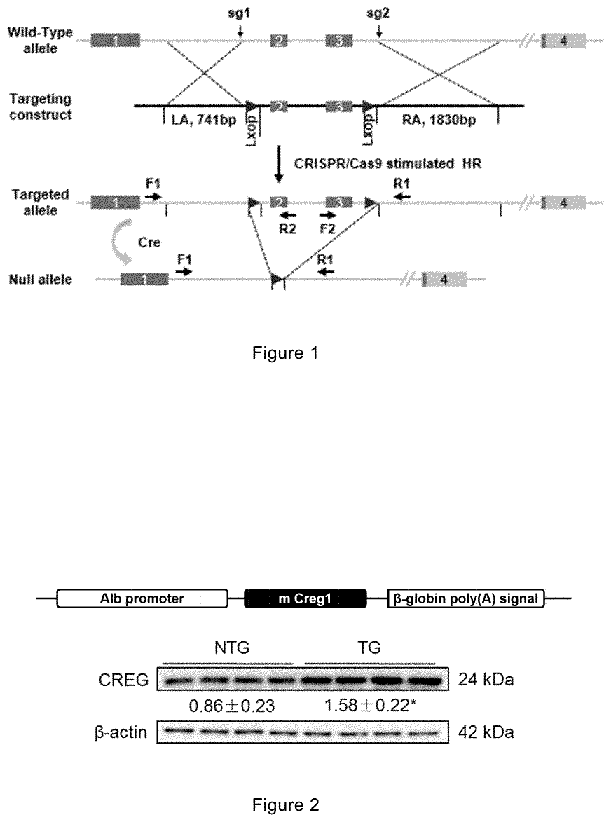

Based on the genetic information, a CRISPR targeting site was designed using CRISPR Design (http://crispr.mit.edu/) in introns 2 and 4, respectively. In addition, a Donor Vector for homologous repair was designed, which includes two side homologous arms, intermediate exons 3 and 4, and two loxp sequences in same direction. FIG. 1 shows a targeting strategy for constructing CREG conditional knockout mice.

1.2 Experimental Process

Construction of targeting vector: Two primers corresponding to sgRNA1 and sgRNA2 were fused into double-stranded DNA, respectively, and then ligated into a pUC57-sgRNA vector digested with restriction endonuclease Bsal through T4 DNA ligase, and the resulted vector could be used for a subsequent in vitro transcription experiment.

1.3 Construction of Donor Vector

The conditional knockout backbone vector pBluescript SK(+)-2loxp constructed in our laboratory contained two loxp sequences in same direction and contained a plurality of restriction endonuclease cleavage sites between the two loxp sequences and on both sides thereof, so as to facilitate clone selection. The CREG conditional knockout donor vector is constructed based on the backbone vector.

Based on the design principles of primers, the following primers were designed to amplify the left and right homologous arms (LA and RA) and the intermediate exon portions (M) of the donor vector. Then, the three fragments obtained by amplification were respectively ligated into the above-mentioned backbone vector by three digestion and ligation steps.

1.4 In Vitro Transcription of Targeting Vectors

The CRIPR/Cas9 system comprises two parts: the Cas9 protein responsible for cleavage and the gRNA that directs the Cas9 protein to localize to the target site. In this experiment, these two parts need to be separately transcribed in vitro. For the Cas9 protein, we digested its expression vector (pST1374-Cas9) with PmeI, purified and recovered the linearized plasmid as a transcription template, and then used a T7 mMESSAGE mMACHINE kit (AM1345, Ambion) to carry out in vitro transcription experiments, so as to obtain the capped mRNA product. A tail was added to the above product using a Poly(A) Tailing kit (Ambion) to obtain a mature mRNA product. For sgRNA, in vitro transcription was performed using a MEGAshortscript™ Kit (AM1354, Ambion). The mRNAs of Cas9 and sgRNA obtained by transcription were purified using a miRNeasy Micro Kit (Qiagen, 217084).

1.5 Microinjection

3-4 Female mice aged 4-5 weeks were selected and intraperitoneally injected with PMSG (30 IU) at 11:00 am on the first day, subsequently injected with hCG (30 IU) after 46-48 hours, and then mated with normal SD male mice. In the next morning, those with vaginal plug after examination were chosen as donor female mice. On the fourth day, at 9:00 am, the donor female mice were sacrificed by cervical dislocation and the fertilized eggs were taken for microinjection. The three kinds of mRNA and the donor plasmid were mixed in a certain ratio to prepare a mixture solution, which was injected into the fertilized eggs of mice by a microinjector (FemtoJet 5247 microinjector, purchased from Eppendorf). After the injection, the fertilized eggs were transplanted into surrogate mice by embryo transplantation, and the pregnant female mice will give birth to offspring within 21-23 days.

1.6 Screening of Founder Mice

The toe or tail tissue of the mice after one week of birth was taken out and the genome was extracted. The two loxp insertion sites in the genome of the newborn mice were separately tested for typing. If homologous recombination occurred, due to a part of the sequence in the genome was replaced by loxp in the donor vector, it differed from the original sequence size and could be resolved by high concentration (3.0%) agarose gel electrophoresis. Accurate homologous recombination repair occurred in all six founder mice. One of the above-mentioned founder mice was randomly selected to mate with a breed conservation C57/B6 mouse in our laboratory, and a F1 generation was obtained after breeding. Heterozygous mice were screened by PCR, and the heterozygotes were mated between each other and propagated. CREG-floxed homozygous mice were finally obtained by the same screening method.

List of primers

| | | | |
|---|---|---|---|
| Se- | pcag-seq-F: CATGTCTGGATCGATCCCCG | | |
| quenc- | pcag-seq-R: CCCTTGCTCCATACCACCCC | | |
| ing | | | |
| primer | | | |
| Full- | AGCTTTGTTTAAACGCCACCATGGCTGCCCGT | PmeI | 663 |
| length | GCTC | | |
| primer | CTAGCTAGCTCACTGCAGCGTGACGTTAAAAT | NheI | bp |
| | ATTC | | |
| CREG-F | CCCCCTGAACCTGAAACATA | | |
| CREG-R | TCAGCGTAGCCTCTGGATTT | | 660 |
| | | | bp |

2. Construction of Hepatocyte-Specific CREG Transgenic (TG) Mice

To further investigate the effects of CREG overexpression on hepatic ischemia-reperfusion injury, we constructed several hepatocyte-specific CREG transgenic mice (CREG-TG). The cDNA was inserted into the downstream of the albumin promoter, and the constructed vector was used by microinjection to construct a fertilized embryo (C57BL/6J background), so as to obtain a hepatocyte-specific CREG transgenic mouse. A schematic diagram of CREG transgene overexpression (TG) is shown in the upper panel of FIG. 2.

The expression quantities of CREG protein in the liver of different transgenic mice were identified by Western blotting: the proteins of liver tissues of different transgenic mice were extracted and quantified by polyacrylamide gel electrophoresis (SDS-PAGE).

The expression of CREG in hepatocytes was driven by the albumin (ALB) promoter and the CREG overexpression was verified by Western blotting. To reflect the changes in CREG under pathophysiological conditions, we selected CREG-TG 10 mice. The results of Western blotting and quantitative analysis showed that the expression of CREG in the liver tissue of CREG-TG 10 mice was significantly increased compared with the normal tissues. The results of CREG transgene overexpression test are shown in the lower panel of FIG. 2.

3. Diet Formulas for Experimental Animals High fat diet (HFD) (purchased from Beijing Huafukang Biotechnology Co., Ltd., Cat. No.: D12942): percentage of calories: proteins: 20%; carbohydrates: 20%; fats: 60%, total calorie/mass ratio: 5.24 kcal/g. Low-fat diet (normal chow, NC) was a normal feed (purchased from Beijing Huafukang Biotechnology Co., Ltd., Cat. No. D12450B): percentage of calories: proteins: 20%; carbohydrates: 70%; fats: 10%, total calorie/mass ratio: 3.85 kcal/g.

4. Animal Feeding and Environmental Conditions

All experimental mice were raised in the animal room of the Institute of Cardiovascular Diseases, General Hospital of Shenyang Military Region. Alternate illumination every 12 hours, temperature 24±2° C., humidity 40% to 70%, and the mice were given free access to water and diet.

### Example 1. Preparation of Fatty Liver and Type 2 Diabetes Model in Mice (Diet Induced Obesity, DIO)

(1) Animal grouping: 8 weeks old male WT mice and CREG-KO (or CREG-TG) mice obtained by the above method were given two kinds of special diets, D12942 High fat diet (HFD) and D12450B normal chow (NC), to form 4 groups: namely, WT-NC group, KO-NC group, WT-HFD group, KO-HFD group (or WT-NC group, TG-NC group, WT-HFD Group, TG-HFD group).

(2) Preparation Process of DIO Mice:

Phenotypic correlation analysis was performed on WT and KO (TG) mice to determine the effects of CREG gene on fatty liver and type 2 diabetes. WT mice and CREG-KO (TG) mice, male, 8 weeks old, were selected and fed with two special diets, D12942 high fat diet (HFD) and D12450B low fat diet (normal chow, NC), and thereby forming 4 groups, namely, WT-NC group, KO/TG-NC group, WT/TG-HFD group, and KO/TG-HFD group. The food intake, fasting body weight and fasting blood glucose level of the mice were recorded in detail every week, and the analysis was performed once every 4 weeks. At the 14^{th} week of the experiment, an intraperitoneal glucose tolerance test (IPGTT) was performed to evaluate the glucose tolerance of the mouse body. At the end of the 16^{th} week, the mouse liver was taken out and photographed, and then a part of the liver was placed in a formaldehyde solution or embedded in an O.C.T tissue freezing medium for pathological analysis.

### Example 2. Determination of Body Weight and Blood Glucose Level in Mice

(1) Detection of Fasting Body Weight and Diet Amount in Mice

(i) Fasting: The experimental mice were fasted (free drinking water) at 8:00 am, and the experimental operation started at 2:00 pm.

(ii) Weighing: Weighing was performed at 0, 4, 8 and 12 weeks, respectively, in which a mouse was caught and placed in a plastic keg on a dynamic electronic balance, and its body weight was measured and recorded.

(iii) Detection of diet amount: After the weighing operation is completed, the mice were fed with diets, and the diet amounts for the mice were recorded by a dynamic electronic balance.

(2) Detection of Fasting Blood Glucose Levels in Mice

All mice to be tested were fasted from 8:00 am to 2:00 pm (free drinking water), that is, the experimental operation was started 6 hours after fasting.

(i) Preparation of blood glucose meter: The battery of blood glucose meter (American Johnson & Johnson, ONETOUCH) was checked, the right switch was pressed, and a test strip was correctly placed into the left slot, the screen displayed the number of the code corresponding to the blood glucose test strip, and then displayed a pattern for dropping blood to indicate that the blood glucose meter was ready for test.

(ii) Fixation of mice: The tail of mouse was grabbed by the right hand, a towel was held on the left hand, then the towel was folded in half, the folded place of the towel was held with the thumb and forefinger, the head and body of mouse was wrapped into the towel in the palm, and the tail base of mouse was fixed with the thumb and forefinger.

(iii) Cutting tail: The tail of mouse was cut quickly at 0.1-0.2 cm from the end of the tail by ophthalmic scissors, and the blood droplets flowed out by themselves.

(iv) Test of blood glucose level: The blood droplets were gently touched with the edge of test strip of the blood glucose meter, then the blood immersed into the test strip, and the blood glucose meter displayed the reading after countdown 5 seconds.

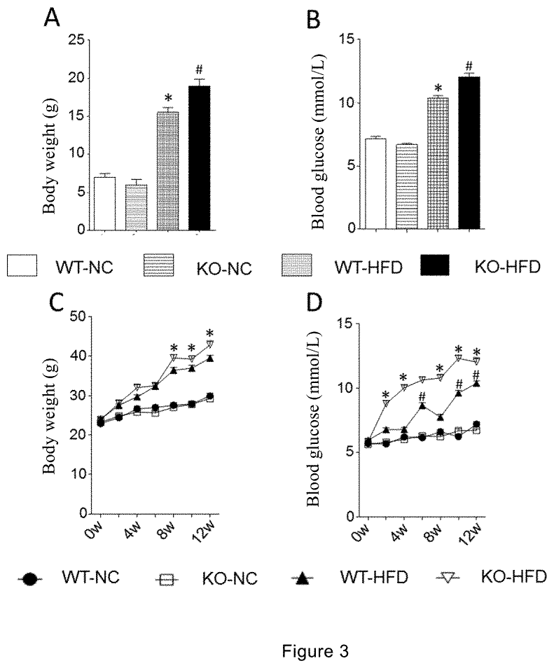

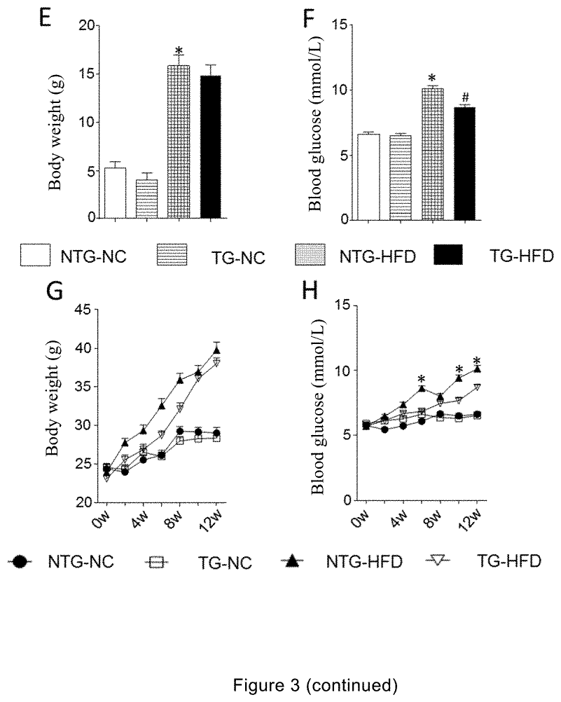

The indicators for assessing the severity of type 2 diabetes included body weight and fasting blood glucose level. The results of changes in body weight and blood glucose level in mice were shown in FIG. 3, in which the WT mice showed a body weight significantly higher than that of the NC group from the 4^{th} week after feeding the HFD (see FIG. 3C), and when the CREG-KO/TG mice were given HFD and NC for 12 weeks, the CREG-KO mice of the HFD group (KO-HFD) showed a body weight significantly higher than that of the WT mice of the HFD group (WT-HFD) from the 4^{th} week to the 12^{th} week (see FIGS. 3C and 3G), while the TG-HFD group with high expression of CREG showed no significant difference in comparison with the NTG-HFD group; it was found in the test of fasting glucose level that the mice in the HFD group showed a fasting blood glucose level significantly higher than that of the corresponding NC control group from the 4^{th}, 8^{th} and 12^{th} week, and the CREG-KO mice in the HFD group also showed a fasting blood glucose level significantly higher than the fasting blood glucose level of the mice of the WT group (see FIG. 3B), while after the high expression of CREG, the blood glucose level of the TG-HFD group was significantly lower than that of the NTG-HFD group (see FIG. 3F). It indicated that the CREG transgenic knockout significantly affects the glucose homeostasis in mice under the HFD feeding condition, and the high expression of CREG gene can significantly increase the ability of glucose metabolism in mice, indicating that the CREG gene plays an important role in type 2 diabetes induced by high fat diet.

### Example 3. Intraperitoneal Glucose Tolerance Test (IPGTT) in Mice

At the 12^{th} week of the experiment, a test of intraperitoneal injection of glucose (IPGTT) was performed to assess the tolerance of the mice to glucose.

(1) Before measuring the blood glucose level, the fasting body weight of the mouse was measured, and the injection volume of glucose was calculated according to 10 μL/g.

(2) The fasting blood glucose level at 0 minutes before the glucose injection was measured, and after the measurement, the glucose solution was rapidly injected intraperitoneally.

(3) Operation Method of Intraperitoneal Injection

(i) Fixation of mouse: the mouse was grabbed, the tail of the mouse was held with the little finger and ring finger of the left hand, while the neck of the mouse was grasped with the other three fingers, so that the head of the mouse was downward, and the abdomen of the mouse was fully exposed.

(ii) Position for inserting needle and injection: the needle was inserted from one side of the abdomen, in which the syringe was held with the right hand, the tip and the mouse abdomen was at an angle of 45°, the needle was inserted, drawn back, and during the injection, the needle passed a small distance under the skin of the abdomen and through the abdomen midline to the other side of the abdomen to enter the abdominal cavity. After the drug was injected, the needle was slowly pulled out and slightly rotated to prevent leakage.

(iii) The tail was cut, and the blood glucose levels were measured at 15 minutes, 30 minutes, 60 minutes, and 120 minutes respectively after intraperitoneal injection in mice, and the blood glucose level values and measurement time were recorded.

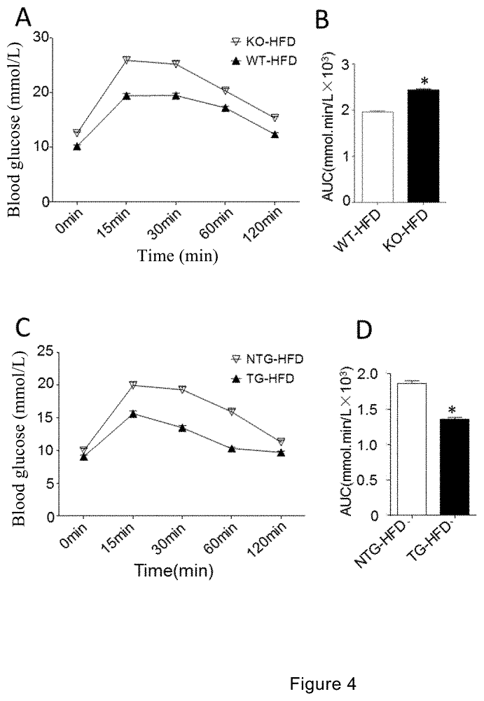

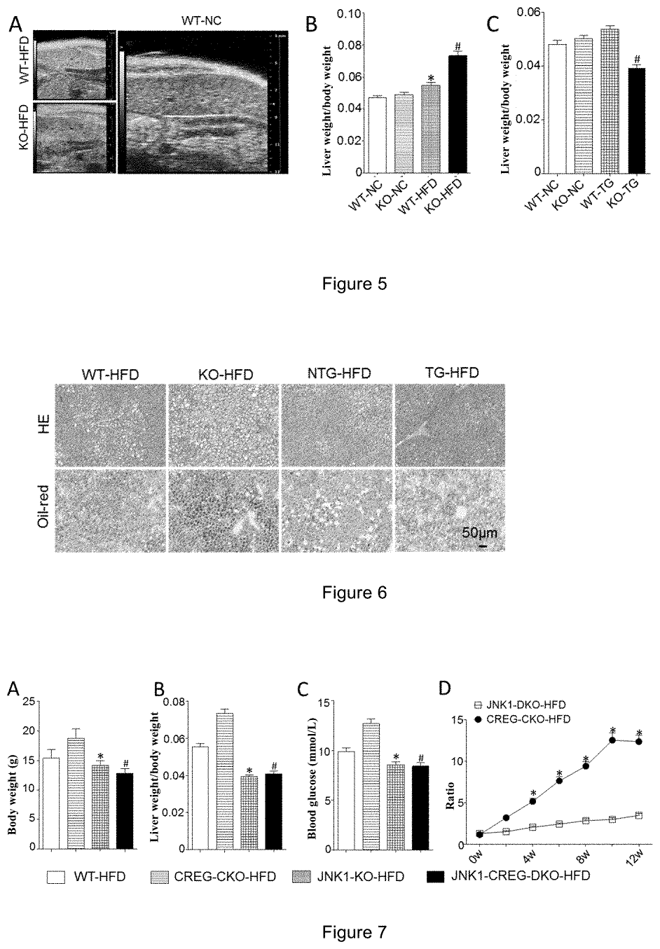

Further, the ability of handling glucose in the mice of each group was evaluated by intraperitoneal glucose tolerance test (IPGTT). At the 12^{th} week of the test, after injection of glucose of 1.0 g/kg body weight, the blood glucose levels of the WT mice and the CREG-KO/TG mice in the HFD group leaped to peak values at a time point of 15 minutes, and the blood glucose levels of the two groups decreased slightly, but were still higher than the fasting blood glucose level (blood glucose at 0 minute), over time to a time point of 60 minutes after injection, and returned to the fasting blood glucose level at 2 hours; additionally, the blood glucose levels of CREG-KO mice were always higher than the blood glucose levels of the WT mice from 0 minute to 2 hours, while the area under the blood glucose curve of the CREG-TG group decreased significantly (see FIGS. 4A and 4C). The above results indicated that the CREG gene plays a vital role in regulating the maintenance of glucose homeostasis.

### Example 4. Insulin Resistance Test in Mice

The detailed operation steps of this example referred to the glucose tolerance test in mice of Example 3. The dosage of insulin was determined by the age and gender of the mice, and an ideal dosage was to reduce the glucose level after 30 minutes of the injection to about 40% of that before the injection. The dosage of insulin used in the insulin resistance test in mice was generally 0.5-1.2 U/kg, and the insulin was diluted with physiological saline, dosage 0.5 U/kg, prepared to have a concentration of 0.5 U/ml. 0.75 U/kg, to measure the blood glucose levels at 0, 15, 30, 60 minutes; 0.027 U/10 g=2.7 U/kg.

The mice were fasted for 4 hours in the morning, normal drinking water. In the afternoon the test was performed, the body weights were measured, the serial numbers were marked, the blood glucose levels were measured before the injection of insulin, and the injection volumes of insulin were calculated on the basis of the body weights. The blood glucose levels were measured at 15 min, 30 min, 45 min, and 60 min, respectively. After the experiment, each cage was supplemented with diets.

The results of the insulin resistance test were shown in FIG. 4B. The results indicated that CREG-KO mice have relatively strong insulin resistance. FIG. 4D shows a significant reduction in insulin resistance in the CREG-TG group.

### Example 5. Determination of Lipid Components in Mouse CREG Liver Tissue

(1) Sampling the Final Liver Tissue

1) After a mouse was weighed, it was quickly sacrificed by cervical dislocation. The mouse was fixed on the back and the hair of its chest and abdomen was moistened with distilled water.

2) The abdomen midline skin of the mouse was clamped using a pair of forceps, and the skin was cut toward the head along the abdomen midline to the xiphoid. Then the skin was cut toward the tail end to expose the subcutaneous fascia, muscles, etc., layer by layer, and the abdominal cavity was opened to fully expose each of organs.

3) The liver of the mouse was quickly found and removed, and the removed liver specimen was placed on sterilized gauze. The residual blood on the surface of the liver was then wiped out. The liver was placed in a sterile Petri dish, quickly photographed and weighed.

4) Freezing specimen: A part of the liver was cut, embedded in a tin foil mold with OCT, frozen and fixed on dry ice.

(2) Frozen Section Treatment of Liver Tissue and Pathological Staining Related Experiments

The changes in total cholesterol content of liver tissue were shown in FIG. 6. After CREG transgenic gene was highly expressed, the total cholesterol content of liver tissue decreased, while the total cholesterol content of liver tissue increased significantly after CREG knockout.

The above results showed that type 2 diabetes and fatty liver disease induced by HFD in the CREG-KO mice were significantly aggravated. These results indicated that the high expression of the CREG gene had a significant effect on the improvement of type 2 diabetes and fatty liver disease. The results of the present invention demonstrated that the CREG gene protein has a potentially important protective effect in disease models of fatty liver and type 2 diabetes.

Although the invention has been described in detail, it will be understood by those skilled in the art that various modifications and alterations can be made in the details of the invention, and they are all within the protection scope of the invention. The full scope of the invention is given by the following claims and any equivalents thereof.

### REFERENCES

1. Arab J P, Karpen S J, Dawson P A, Arrese M, Trauner M. Bile acids &amp; nonalcoholic fatty liver disease: Molecular insights and therapeutic perspectives. Hepatology. 2016 Jun. 30. doi: 10.1002/hep.28709. [Epub ahead of print].
2. Valenti L, Bugianesi E, Pajvani U, Targher G. Nonalcoholic fatty liver disease: cause or consequence of type 2 diabetes? Liver Int. 2016 Jun. 8. doi: 10.1111/liv.13185. [Epub ahead of print]
3. Katsiki N, Mikhailidis D P, Mantzoros C S. Non-alcoholic fatty liver disease and dyslipidemia: An update. Metabolism. 2016 August; 65(8):1109-23. doi: 10.1016/j.metabol.2016.05.003. Epub 2016 May 13.
4. Tahrani A A, Barnett A H, Bailey C J. Pharmacology and therapeutic implications of current drugs for type 2 diabetes mellitus. Nat Rev Endocrinol. 2016 Jun. 24. doi: 10.1038/nrendo.2016.86. [Epub ahead of print]
5. Alemán-González-Duhart D, Tamay-Cach F, Álvarez-Almazán S, Mendieta-Wejebe J E. Current Advances in the Biochemical and Physiological Aspects of the Treatment of Type 2 Diabetes Mellitus with Thiazolidinediones. PPAR Res. 2016; 2016: 7614270. doi: 10.1155/2016/7614270. Epub 2016 May 23.
6. Peng C, Pei H, Wei F, Tian X, Deng J, Yan C, Li Y, et al. Cellular repressor of E1A-stimulated gene overexpression in bone mesenchymal stem cells protects against rat myocardial infarction. Int J Cardiol 2015; 183: 232-241.
7. Xu L, Liu J M, Chen L Y. CREG, a new regulator of ERK1/2 in cardiac hypertrophy. J Hypertens 2004; 22: 1579-1587.
8. Bian Z, Cai J, Shen D F, Chen L, Yan L, Tang Q, Li H. Cellular repressor of E1A-stimulated genes attenuates cardiac hypertrophy and fibrosis. J Cell Mol Med 2009; 13: 1302-1313.
9. Sun M, Tian X, Liu Y, Zhu N, Li Y, Yang G, Peng C, Yan C, Han Y. Cellular repressor of E1A-stimulated genes inhibits inflammation to decrease atherosclerosis in ApoE(−/−) mice. J Mol Cell Cardiol 2015; 86: 32-41.
10. Liu Y, Tian X, Li Y, Liu D, Liu M, Zhang X, Zhang Q, Yan C, Han Y. Up-Regulation of CREG Expression by the Transcription Factor GATA1 Inhibits High Glucose- and High Palmitate-Induced Apoptosis in Human Umbilical Vein Endothelial Cells. PLoS One 2016; 11: e0154861.
11. Deng J, Han Y, Sun M, Tao J, Yan C, Kang J, Li S. Nanoporous CREG-eluting stent attenuates in-stent neointimal formation in porcine coronary arteries. PLoS One 2013; 8: e60735.
12. Han Y, Guo L, Yan C, Guo P, Deng J, Mai X, Kang J, Li S. Adenovirus-mediated intra-arterial delivery of cellular repressor of E1A-stimulated genes inhibits neointima formation in rabbits after balloon injury. J Vasc Surg 2008; 48: 201-209.
13. Li Y, Yan C H, Han Y L. CREG mediated adventitial fibroblast phenotype modulation: a possible therapeutic target for proliferative vascular disease. Med Hypotheses. 2012 July; 79(1): 95-7.

1.-4. (canceled)

5. A composition comprising a CREG protein or an active fragment thereof, a nucleic acid molecule encoding a CREG protein or an active fragment thereof, a recombinant vector or recombinant cell expressing a CREG protein or an active fragment thereof, and optionally a pharmaceutically acceptable carrier or excipient, wherein the composition is for at least one of lowering blood glucose level, treating insulin resistance, and reducing total cholesterol content of the liver.

6. A kit comprising an agent for detecting an expression level of a CREG protein or an active fragment thereof, wherein the kit is for the prediction and/or evaluation of treatment effect and prognosis of at least one of blood glucose level, insulin resistance, and total cholesterol content of the liver.

7.-9. (canceled)

10. A CREG protein or an active fragment thereof at least one of lowering blood glucose level, treating insulin resistance, and reducing total cholesterol content of the liver.

11.-12. (canceled)

13. A nucleic acid molecule encoding a CREG protein or an active fragment thereof and a downstream regulatory target gene thereof, and a recombinant vector or recombinant cell expressing a CREG protein or an active fragment thereof at least one of lowering blood glucose level, treating insulin resistance, and reducing total cholesterol content of the liver.

14. The nucleic acid molecule encoding a CREG protein or an active fragment thereof, and a downstream vector thereof, a recombinant vector or recombinant cell expressing a CREG protein or an active fragment thereof, according to claim 13, wherein the downstream regulatory target gene is JNKI.

15. (canceled)

16. A method for lowering blood glucose level, treating insulin resistance, and/or reducing total cholesterol content of the liver, comprising administering to a subject a therapeutically effective amount of a CREG protein or an active fragment thereof.

17. The method of claim 16, comprising administering to the subject a therapeutically effective amount of a nucleic acid molecule encoding a CREG protein or an active fragment thereof, and a downstream regulatory target gene, a recombinant vector or recombinant cell expressing the CREG protein or an active fragment thereof.

18. The method of claim 16, comprising administering to the subject a therapeutically effective amount of a substance that modulates the expression level of the CREG gene or CREG protein.

19. The method of claim 17, wherein the downstream regulatory target gene is JNKI.

20. The method of claim 16, wherein the subject is a mammal, optionally the subject is selected from the group consisting of a rat, a mouse, a dog, a pig, a monkey, a human.


SEQUENCE LISTING (6 sequences)

SEQ ID NO: 1 - DNA, 20 bp, Artificial Sequence
  Artificial sequencing primer pcag-seq-F
catgtctggatcgatccccg

SEQ ID NO: 2 - DNA, 20 bp, Artificial Sequence
  Artificial sequencing primer pcag-seq-R
cccttgctccataccacccc

SEQ ID NO: 3 - DNA, 36 bp, Artificial Sequence
  Artificial full-length primer with PmeI site
agctttgtttaaacgccaccatggctgcccgtgctc

SEQ ID NO: 4 - DNA, 36 bp, Artificial Sequence
  Artificial full-length primer with NheI site
ctagctagctcactgcagcgtgacgttaaaatattc

SEQ ID NO: 5 - DNA, 20 bp, Artificial Sequence
  Artificial primer CREG-F
ccccctgaacctgaaacata

SEQ ID NO: 6 - DNA, 20 bp, Artificial Sequence
  Artificial primer CREG-R
tcagcgtagcctctggattt